(19) United States
(12) Patent Application Publication
(10) Pub. No.: US 2003/0127603 A1
(43) Pub. Date: Jul. 10, 2003

(54) Apparatus for the inactivation of pathogens in protein-containing fluids and uses thereof

(76) Inventors: Bernard Horowitz, Key Biscayne, FL (US); Xiaoliang Wang, Pinebrook, NJ (US); Lawrence D. Barr, Tucson, AZ (US)

Correspondence Address: CLARK & ELBING LLP, 101 FEDERAL STREET, BOSTON, MA, 02110, US

(21) Appl. No.: 10/147,002

(22) Filed: May 15, 2002

Related U.S. Application Data
(60) Provisional application No. 60/291,108, filed on May 15, 2001.

(51) Int. Cl. A61L 2/00

(52) U.S. Cl. 250/435; 422/24

(57) ABSTRACT

The invention features apparatuses and methods for inactivating viruses, bacteria, and other pathogens in protein-containing fluids. The apparatuses include a radiation transparent housing at least of a portion of which is radiation transparent. In addition, the housing contains a static mixer such that fluid flowing the housing approximates fully mixed flow. Pathogens are inactivated in liquids flowing through an apparatus of the invention by exposing the liquid to UV radiation, e.g., UVC radiation, at an appropriate intensity for an appropriate period of time.

### CROSS-REFERENCE TO RELATED APPLICATIONS

This application claims priority from U.S. Provisional Application No. 60/291,108, filed May 15, 2001, hereby incorporated by reference.

### BACKGROUND OF THE INVENTION

The invention is directed to the inactivation of pathogens in protein-containing compositions.

The widespread application of specific virus inactivation techniques such as solvent/detergent, pasteurization, and dry-heat treatment has greatly increased the safety of plasma products. Each of these techniques is, however, only partially effective in inactivating non-enveloped viruses and heat-stable viruses. Irradiation with ultraviolet light having a wavelength between 240 and 280 nm (UVC irradiation) has been advocated as a supplemental method to present technologies because of its universal virucidal effects. Moreover, UVC treatment is theoretically capable of inactivating all pathogens that contain nucleic acids.

The commercialization and utilization of UVC-mediated viral inactivation has been slowed by the fact that the presence of desirable therapeutic proteins in plasma and plasma products results in a lower transmission at the UVC irradiation wavelength. For example, the transmission of 254 nm light in human plasma is only about 20% at a depth of 0.2 mm. Because of this low transparency of proteins, most early designs focused on the use of thin films. Different designs such as the LoGrippo flat quartz cell and the Dill irradiator (in which sample is pumped onto the inside top of a rotating stainless steel cylinder) have been developed in the past decades using this thin film design. Prior experimentation with the Dill irradiators shows that viral inactivation in liquids can be successful when a thin film of liquid is exposed to UV irradiation for a sufficient time to ensure that all flow elements are irradiated. Observed differences in flow stream depth in Dill irradiators indicate, however, that increased UV irradiation intensity or exposure time would be required to achieve the same amount of pathogen activation for all flow streams compared to a device that fully mixes fluids. This increased irradiation would result in a reduction in preservation of protein biological activity.

Thus, there is a need to develop better methods and apparatuses for the inactivation of pathogens, especially non-enveloped viruses and heat-stable viruses.

### SUMMARY OF THE INVENTION

The invention features apparatuses and methods for inactivating viruses, bacteria, or other pathogens in protein-containing fluids.

In one aspect, the invention features an apparatus for treating a liquid with UV radiation, e.g., UVC radiation, to inactivate pathogens contained in the liquid. The apparatus includes a housing having a liquid flow path and a static mixing device located therein. At least a portion of the housing is radiation-transparent and adjacent to a source of UV radiation that is adapted to irradiate the mixed liquid at an intensity and for a duration to inactivate pathogens in the liquid. In various embodiments, the housing may include a radiation-transparent plate, two radiation-transparent plates, or a radiation transparent cylinder. The radiation-transparent housing may be flanked by at least two sources of UV radiation. In various embodiments where the radiation-transparent housing is cylindrical, the housing may be flanked by at least three or even four sources of UV radiation. For a two-plate housing, the plates may be substantially parallel (e.g., ±10 μm when the inner surfaces are 0.4 mm apart) and the inner surfaces of the plates are about 0.01 to 2 mm from each other. In one embodiment, the inner surfaces of the plates are about 0.4 to 1 mm from each other. In another embodiment, the inner surfaces of the plates are about 0.4 to 2.0 mm from each other. In another embodiment, the inner surfaces of the plates are about 0.4 to 0.75 mm from each other.

In another aspect, the invention features a method for inactivating pathogens in a liquid. The method includes providing an apparatus for treating a liquid with UV radiation, e.g., UVC radiation, to inactivate pathogens contained in the liquid, as is described above; passing the liquid through the static mixing device; and irradiating at least a portion of the liquid with UV radiation while the liquid is within the housing, wherein the irradiating inactivates at least some of the pathogens in the liquid. The method may include additional pathogen inactivation steps, such as performing solvent/detergent extraction on the liquid after irradiation or performing pathogen inactivation steps, using pathogen-inactivating compounds, e.g., aziridino compounds, such ethyleneimine oligomers, on the liquid after irradiation. The flow rate of the liquid through the apparatus is, for example, at least 10 ml/min, at least 30 ml/min, at least 100 ml/min, or at least 200 ml/min.

In various embodiments of either of the above aspects, the UV radiation reduces the number of infective pathogens in the liquid by at least 3 logs. In other embodiments the number of infective pathogens in the liquid are reduced by at least 4 log, 5 logs, 6 logs, 7 logs or greater than 7 logs. The proteins of the pathogen-inactivated liquid may retain, for example, at least 75%, 80%, 85%, 90%, or greater of the activity of a protein relative to the activity of that protein in the liquid prior to pathogen inactivation. The static mixers of the invention cause the liquid flowing through the apparatus to exhibit a flow pattern approximating fully mixed flow. The percentage of fully mixed flow may be, for example, approximately 70%, 80%, 90%, 95% or greater.

Exemplary proteins include Factor V, Factor VII, Factor VIII, Factor IX, Factor XI, Factor XIII, AT-3, thrombin, fibrinogen, prothrombin, IVIG, alpha-1 proteinase inhibitor, and albumin. Exemplary liquids include plasma, antihemophilic factor concentrate, prothrombin complex concentrate, and intravenous immunoglobulin. The liquid may include a free radical scavenger, e.g., rutin or tryptophan. In other embodiments, the pathogen is a virus, e.g., encephalomyocarditis virus, parvovirus, hepatitis A virus, hepatitis B virus, hepatitis C virus, human immunodeficiency virus, pseudorabies virus, or herpes virus.

By “intensity” is meant the strength of irradiation, expressed as mW/cm².

By “fluence” is meant the dosage of radiation, calculated from intensity (W/cm²)×resident time (seconds) and expressed as J/cm².

By “local fluence” is meant the fluence at a given point.

By “log of EMCV” or “log of virus” is meant the base 10 logarithm of the number of infectious viral particles, as assayed by standard means.

By “EMCV kills (log)” or “viral kill (log)” is meant the base 10 logarithm of the change in the number of viral particles, as assayed by standard means, after inactivation.

By “static mixer” is meant a motionless device for mixing fluids.

By “approximating fully mixed flow” is meant having a flow profile in which the fluid streams have been disrupted to an extent that individual streams spend an approximately equal amount of time at every depth in a container, e.g., a cylindrical tube or a flat-plate chamber. The exact amount of mixing required depends on the liquid being mixed, the illumination, and the characteristics of the irradiation chamber. One skilled in the art can make this determination.

Other advantages and features of the invention will be apparent from the following description and the claims.

### BRIEF DESCRIPTION OF THE DRAWINGS

FIG. 1 is a schematic illustration depicting the cell model and illumination geometry.

FIGS. 2 and 3 are graphs showing the predicted local pathogen survival probability and local protein survival probability as a function of the position of the pathogen or protein in the cell following single-sided UVC illumination of plasma. Cell height is 0.02 cm (FIG. 2) or 0.04 cm (FIG. 3), illumination (I=8.3×10⁻³ W/cm²) is from the left.

FIGS. 4 and 5 are graphs showing the predicted total pathogen survival probability and total protein survival probability as a function of the position of the pathogen or protein in the cell following single-sided UVC illumination of plasma. In FIG. 4, modeling was performed with Bₚ (the rate constant for UVC irradiation-mediated modification of a protein) at 1.7, 5.1, and 17 J⁻¹ cm² and a channel height of 0.01 to 0.04 cm, while in FIG. 5, the results from one rate constant (5.1 J⁻¹ cm²) are depicted for channel heights of 0.02 to 0.04 cm.

FIG. 6 is a graph showing the predicted local pathogen survival probability and local protein survival probability as a function of the position of pathogen or protein in the cell following double-sided UVC illumination of plasma. Cell height is 0.04 cm. The illumination from each irradiator is half that used in the modeling depicted in FIGS. 2-5 (i.e., I=2×4.15×10⁻³ W/cm²).

FIGS. 7 and 8 are graphs showing the predicted total viral survival probability and total protein survival probability as a function of cell height following double-sided UVC illumination of plasma at a channel height of 0.01 to 0.04 cm.

FIG. 9 is a graph showing the predicted total viral survival probability and total protein survival probability as a function of cell height following single-sided UVC illumination of well-mixed plasma.

FIGS. 10 and 11 are graphs showing total viral survivability as a function of total protein survivability for three values for Bₚ (5.1, 6.12, and 7.34 J⁻¹ cm²) and two values of Bᵥ (FIG. 10, 384 J⁻¹ cm²; FIG. 11, 461 J⁻¹ cm²) following single-sided illumination of well-mixed plasma.

FIGS. 12A and 12B are schematic illustrations showing an exemplary static mixing device for use in the flat plate irradiator of the invention. In this static mixing device, a mesh 2 is oriented such that the wires (represented by black and white bars) are at a forty-five degree angle to the flow path in the channel in the absence of the static mixing device. On one the front and back surfaces of the mesh are barrier strips 6 and 4 (represented by the hatched gray bars and the gray bars, respectively), also oriented at a forty-five degree angle to the flow path in the channel in the absence of the static mixing device such that the barrier strips 6 on the front surface are perpendicular to the barrier strips 4 on the back surface. The illustrations are not drawn to scale.

FIG. 13 is a series of side and sectional views of an entry side exposure quartz plate of the flat plate irradiator assembly of the invention.

FIG. 14 is a series of side and sectional views of an exit side quartz exposure plate of the flat plate irradiator assembly of the invention, additionally showing a plan view of said plate.

FIG. 15 is a plan view of one of two identical exposure plate retainers of the flat plate irradiator apparatus of the invention, showing attachment points, and a pair of end views of said exposure plate retainer.

FIG. 16 is a plan view of an upper pressure plate of the flat plate irradiator apparatus of the invention, showing attachment points.

FIG. 17 is a plan view of a lower pressure plate of the flat plate irradiator apparatus of the invention, showing attachment points.

FIG. 18 is a plan view of a load spreader plate of the flat plate irradiator assembly of the invention.

FIG. 19 is a section, A-A, of the load spreader of FIG. 18.

FIG. 20 is a set of views of a connector port of the flat plate irradiator apparatus of the invention.

FIG. 21 is a plan view of a shim of the flat plate irradiator apparatus of the invention.

FIG. 22 is a graph of the change in uridine 5′-monophosphate (UMP) concentration in a cylindrical irradiator as a function of the time a lamp is operated at a flow rate of 15 ml/min.

FIG. 23 A-E is a series of illustrations of various schemes for cylindrical irradiators. The relative location of lamps 100 and tubes 102 are shown.

### DETAILED DESCRIPTION OF THE INVENTION

We have invented irradiators for the inactivation of viruses, bacteria, or other pathogens in liquids containing UV-sensitive biomolecules such as proteins. As is described below, the irradiators of the present invention provide an approximately uniform exposure of liquids to UV light. The irradiators include UV transparent chambers through which a liquid flows. Static mixers may be employed in the chambers to allow each flow element to pass near the surface of the chamber for approximately the same length of time. In this manner, the entire flowing stream of a liquid receives approximately equal exposure to UV radiation. Methods that use these irradiators to inactivate pathogens in liquids containing biomolecules, such as proteins, have also been developed.

Irradiators

Any chamber that has a UV transparent window may be used in the methods of the invention. Examples of irradiators include those employing flat plate or cylindrical designs. Advantageously, the chambers contain static mixers so that the flow streams of a moving fluid receive approximately equal exposure to UV radiation. Desirably, irradiation occurs during liquid passage through a static mixing device, but irradiation may also occur before or after passing through the static mixer. If desirable, the static mixing device can be modified to increase shearing of the liquid as it passes through the mixer. Examples of static mixing devices are provided herein. Irradiation can be from one or more sides. Light baffles can be used to restrict the area of the plates to be exposed to UV irradiation. An air circulation unit may also be used to remove excess heat from the UV sources. Other cooling devices, e.g., water-based coolers, are known in the art. The liquid may be passed through a single irradiator several times or through a series of irradiators in order to achieve the desired degree of pathogen reduction.

Static Mixers

One skilled in the art will recognize that numerous static mixing devices can be used in the present invention, so long that the mixer disrupts the liquid flow such that it approximates a fully-mixed liquid. For example, meshes are available in a variety of gauges (representing the number of wires/inch) and wire thicknesses. Means for determining whether a static mixing device provides adequate liquid mixing are well known in the art. In one embodiment, the static mixing device allows for at least a 4-log reduction in infectious pathogens without more than a 20% loss in activity of a blood coagulation protein.

Static mixing devices are available in a variety of materials (e.g., stainless steel, Teflon™, copper). In selecting materials for use in the viral inactivating apparatus of the present invention, it is desirable to select materials that will not react with or cause reactions in components of the liquid to be irradiated. It is also desirable that the material be durable and amenable to sterilization (e.g., by autoclaving or chlorine treatment), especially if the treated liquid is to be administered to a human or other animal.

Static mixers may also include a physical barrier to prevent a fluid from flowing in a region of a chamber that receives inadequate illumination, e.g., the central 2 mm of a 5-mm diameter cylindrical tube. A static mixer may be opaque or transparent.

Liquids

The methods and apparatuses of this invention provide protein-containing compositions, for example, blood cell derivatives (e.g., hemoglobin, alpha interferon, human growth hormone, erythropoietin, PDGF, tPA, etc.), blood plasma, blood plasma fraction (e.g., fresh frozen plasma, thawed frozen plasma, cryoprecipitate, cryosupernatant, ethanol supernatant or polyethylene glycol supernatant), or a product derived therefrom, which are rendered essentially free of pathogens while retaining at least 75% (e.g.,>80%) of the activity of a protein present before irradiation.

The liquids treated may be any liquid obtained from any animal, e.g., a mammal such as a human. Such liquids include, e.g., plasma, plasma fractions, plasma concentrates, and components thereof. The process, however, is also useful in treating cell lysates or proteins secreted by cells. Also contemplated are the treatment of fractions derived from platelets, white cells (leukocytes), red blood cells, fibroblasts, and solutions of interferon, growth hormone, tPA, Factor VIII, transfer Factor, hemoglobin, growth factors, EPO, or DNAse.

Pathogen Inactivation

Pathogens present in products of normal or transformed cells can be inactivated using the methods and apparatuses described herein while retaining a desired protein activity, e.g.,>75%, in such products. For example, one can inactivate products produced using normal or transformed cells, hybridomas, or genetic engineering.

Pathogens that can be inactivated by the methods and apparatuses of the present invention include bacteria, viruses, or other blood-borne parasites. Exemplary viruses include, for example, human immunodeficiency viruses types 1 and 2 (HIV-1 and HIV-2), human T-cell lymphotropic virus type I (HTLV-I), human T-cell lymphotropic virus type II (HTLV-II), cytomegalovirus (CMV), hepatitis A virus (HAV), hepatitis B virus (HBV), hepatitis C virus (HCV), hepatitis G virus (HGV; also known as hepatitis GB virus, type C (HGBV-C)), bovine viral diarrheal virus (BVDV), parvovirus (e.g., porcine parvovirus, parvovirus B19), encephalomyocarditis virus (EMCV), pseudorabies virus, vesicular stomatitis virus, and herpes virus. Exemplary bacteria include, for example, Yersinia enterocolitica and Treponema pallidum. Blood-borne parasites include, for example, Plasmodium spp. (e.g., P. falciparum), Trypanosoma cruzi, and Leishmania spp. (e.g., L. donovani).

The amount of infectious pathogen is reduced, for example, by at least 3, 4, 5, 6, 7 logs, or more.

Protein Viability

In addition to inactivating pathogens, UV radiation may also initiate undesirable modifications of proteins. Damage to proteins by UVC involves photoionization of amino acids as the most important primary reaction. Different proteins have different amino acid constituents, especially those situated in active sites. Thus, the sensitivity of a given protein (e.g., different clotting factors) to UVC varies. The recovery varies from about 55% to 80% depending on the irradiator design employed. Designs that include mixers achieve greater recovery of proteins.

Damage to plasma proteins by UVC also involves another important aspect, various free radical reactions. Therefore, in some embodiments, the addition of a free radical scavenger or quencher, e.g., rutin, may greatly enhance the recovery of proteins from irradiated liquids. For example, recovery of Factor VIII in AHF increased from about 30% to 95% with the addition of rutin in a cylindrical irradiator, and the recovery of several clotting factors in plasma increased from about 60% to 75%. Other quenchers include, for example, tryptophan, propyl gallate, catechin, copper ascorbate, β-carotene, and uric acid.

The pathogen-inactivated liquid retains at least 75%, 80%, 85%, 90%, or greater of the activity of a protein, compared to the activity of that protein present in the liquid before the liquid is UV-irradiated. Exemplary proteins whose activity is retained include, for example, Factor V, Factor VII, Factor VIII, Factor IX, Factor XI, Factor XIII, AT-III, thrombin, fibrinogen, prothrombin, alpha-1 proteinase inhibitor, and albumin. The liquid can be, for example, plasma, antihemophilic factor concentrate (AHF), prothrombin complex concentrates (PCC), or intravenous immunoglobulin.

Flat Plate Irradiator

We have invented a flat plate irradiator for the inactivation of viruses, bacteria, or other pathogens in liquids containing UV-sensitive biomolecules such as proteins. As is described below, the flat plate irradiator of the present invention spreads the liquid flow stream to approximate a uniform, controlled-depth film prior to and during UV exposure. The flat plate irradiator assembly includes two plates, at least one of which is UV-transparent, with a machined shim plate and a static mixing device placed between the plates. The plates form a shallow flow channel with depth governed by the shim plate thickness. Liquid enters the assembly at the entry port, then through a distribution channel that is substantially less resistant than the shallow flow channel. From there, liquid passes into the shallow space between the plates, through the static mixing device, and out through an exit port. Shim plates may be made in numerous thicknesses and with any of a variety of central openings to provide for multiple flow channel configurations. The static mixing device can be, for example, a woven mesh having a thickness essentially identical to the flow channel depth. Liquid passing in the spaces between the mesh becomes divided into multiple streams, which forces viruses or other pathogens to pass near the plate surfaces and ensures adequate UV exposure. Several flat plate irradiators may be used in sequence or in parallel.

The Radiation Core. The radiation core is a “sandwich” assembly consisting of two flat plates, one or both of which may be UV transparent (e.g., quartz (crystalline or amorphous)), clamped together with a spacer shim between. Hardware is applied to clamp the plates to form a watertight seal. The UV transparent flat plates can be fabricated, for example, from UV grade quartz that transmits more than 90% of radiation at 254 nm. In usage, the core “sandwich” assembly is mounted in a holder inside a protective enclosure with a UV lamp on one or both sides. Liquid is pumped into the core through an entry connector port and passes between the two plates to an exit connector port similar to the entry port. In one embodiment, the UV exposure surface area is 2×8 inches. The flow depth depends on the spacer thickness, ranging from 0.01 to 2 mm (e.g., from 0.1 to 1 mm).

In order to maintain substantially uniform flow velocity through the space between the two plates, a high degree of flatness is desired in the two parallel surfaces defining this enclosure. In the absence of the static mixing device, laminar flow will exist and the basic governing equation for flow through a rectangular orifice is given below.

$$U = \frac{Δ P}{2 μ L} \times ( \frac{{h}^{2}}{4} - {y}^{2} )$$

Where:

U=flow velocity at point y within the orifice (y varies between ±h/2)

ΔP=pressure drop across orifice

h=orifice height

L=orifice length

μ=liquid viscosity

This equation shows a parabolic flow velocity distribution as a function of depth y, with maximum flow velocity occurring at y=0 (the center of the orifice) given by:

$$U = \frac{Δ P \cdot {h}^{2}}{8 μ L}$$

Average flow velocity through the orifice is 2/3 the maximum velocity for parabolic distribution. The volumetric flow through the orifice is the average velocity multiplied by the cross-sectional area of the enclosure, or:

$$Q = \frac{Δ P \cdot b \cdot {h}^{3}}{12 μ L}$$

Where b=the width of the orifice, assumed to be much greater than the orifice height so that end effects are negligible.

The maximum velocity through the gap or channel depends on the square of the gap height (h). Since maximum flow velocity through the channel for a given volumetric flow determines the UV exposure time, it is desirable to keep the variation in channel height to a minimum. Taking as an example a 400 μm channel height, and allowing no more than 10% velocity variation through the channel, leads to the determination that the surfaces must be flat and parallel to each other within about 20 μm. Using half this value to allow for error during fabrication means that each plate should have one surface polished flat within 5 μm. This degree of flatness can be readily obtained using standard methods.

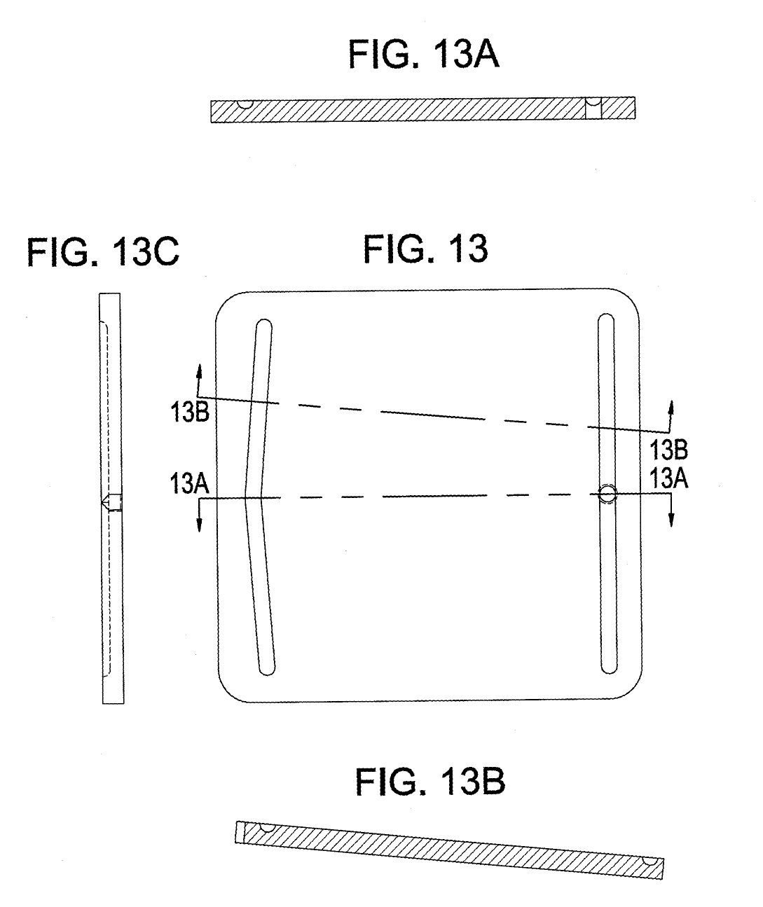

While the orientation of the plates relative to gravity is not significant in terms of the ability of the irradiator to inactivate pathogens in the liquid, we have found through testing that an important secondary consideration is the ability to remove trapped air from the closed apparatus. For this reason, the apparatus described in FIG. 13 includes plates oriented vertically, with the liquid flowing from bottom to top. One skilled in the art will recognize that other methods for preventing or removing trapped air can also be used in conjunction with the present apparatus.

Thermal Control. As is described herein, the UV transparent plates may be quartz. Although quartz has a low coefficient of thermal expansion (0.55×10⁻⁶/° C.), it is desirable to include consideration of thermal expansion in the design of a flat plate irradiator, and to include means for thermal regulation.

There are at least two heat sources to be considered in regards to the quartz plates: the heat from the UV radiation sources and that resulting from UV absorption by the plates. If there is a temperature difference between the two surfaces of the plate, the plate will distort slightly. The magnitude of distortion is measured as a change in gap height and can be calculated by:

$$Δ h = \frac{{W}^{2} \cdot α \cdot Δ T}{4 t}$$

Where:

W=maximum span across the quartz plate

t=plate thickness

α=coefficient of thermal expansion

ΔT=temperature gradient through the plate thickness

Δh=change in gap height due to temperature gradient

The foregoing equation can be used to determine the ΔT allowable to achieve a desired flatness. For example, to achieve a Δh of 5 μm with a plate having the dimensions of 250 mm×250 mm×12.5 mm, a maximum allowable ΔT is calculated to be about 3.7° C.

Static Mixing Devices. The flow in the flat plate irradiators of the invention is laminar. Thus, a particle suspended in the liquid will remain at the same depth (relative to the surface of the plate) throughout its transit time through the cell. Because more irradiation is adsorbed by the plasma as depth increases, an important corollary is that for two particles at different depths, the one closer to the plate surface will receive greater irradiation than the one farther from the plate. This irradiation pattern is true for both proteins and pathogens. The use of chambers having a thick shim (e.g., 1 mm) but without a mixer is precluded in instances of laminar flow because the amount of energy required to inactivate pathogens at depths far from the surface of the plate will result in an unacceptable amount of protein modification, as determined by coagulation activity.

The foregoing problem is made worse by the fact that, in laminar flow through a narrow rectangular channel, the flow velocity is parabolic as a function of height. This flow is characterized by the formula: v(x)=v(0) [1−(x/xₒ)²], where v(0) is the centerline velocity and the volume-averaged velocity, vₐᵥ=2/3 v(0). Thus, the viral particles in the center of the channel receive less irradiation and have a faster transit time through the cell than particles near the edges at the same depth.

In order to overcome the problems associated with laminar flow through an irradiator, we developed static mixing devices that would disrupt the laminar flow and, more importantly, produce flow characteristics approximating fully-mixed flow. The use of a suitable static mixing device (such as one described herein) allows for reduced irradiation and less protein modification for the same amount of viral inactivation.

In one example, a static mixing device was constructed from 26-mesh screen consisting of stainless steel wire having an approximate diameter of 0.015 inches. The thickness of the screen was accordingly about 0.030 inches or 0.75 mm. When the static mixing device is placed between the two plates, it is desirable that it contacts the inner surfaces of the plates. Thus, a shim having the same thickness as the static mixing device is selected (thus, in this case, defining the channel height (h) as 0.30 inches. The static mixing device is oriented such that the mesh wires are at a forty-five degree angle to the flow path in the absence of the mixer.

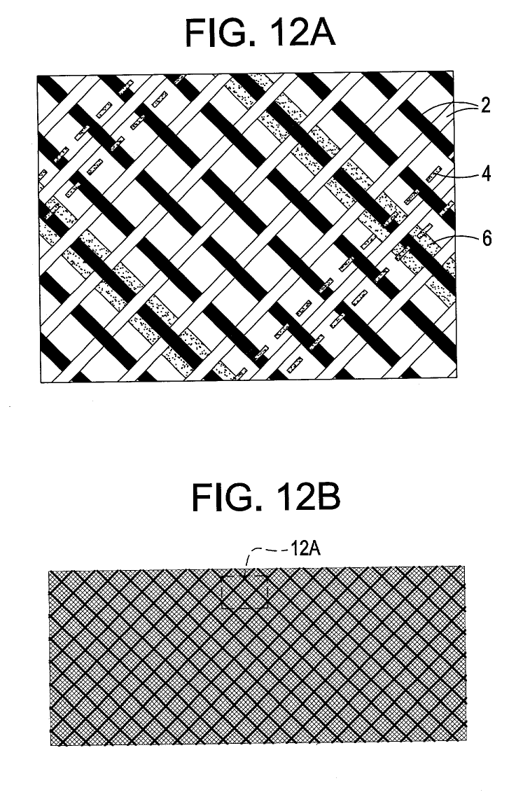

If desirable, the static mixing device can be further modified by the addition of barrier strips. For example, fluid shear resulting from the static mixing device described above can be augmented by the addition of epoxy barrier strips parallel to the mesh wire (thus creating a series of diagonal barriers that do not cross each other), with the barrier strips on one side of the mesh oriented perpendicular to those on the other side (FIGS. 12A and 12B). While the barrier strips can be placed at any interval, it is desirable to place them at intervals between 0.0625 and 0.5 inches. While partially penetrating the mesh, the barrier strips do not add thickness to the static mixing device. When placed between the two plates, the outer surfaces of the strips will thus be flush against the inner plate surfaces.

Double-sided Illumination. When the flow is fully mixed, single-sided illumination is sufficient for the inactivation of pathogens without the modification of coagulation proteins. Double-sided illumination may be used for a better viral activation/protein modification ratio. This effect is demonstrated in the examples described below.

1-D Flow Model. UV irradiation-induced pathogen inactivation and protein inactivation in a flowing liquid medium can be determined using a simple numerical model in which the volume-dependent survival fraction is exponentially dependent on local exposure fluence as follows:

S_{f}(x)=exp[−B I(x)t(x)]

where B is the rate constant for UVC irradiation-mediated inactivation of a species (e.g., a pathogen or a protein) in units of J⁻¹ cm²; I(x) is the local irradiance (W/cm²); and t(x) is the liquid element exposure time as a function of liquid film depth x. The local irradiance is defined as:

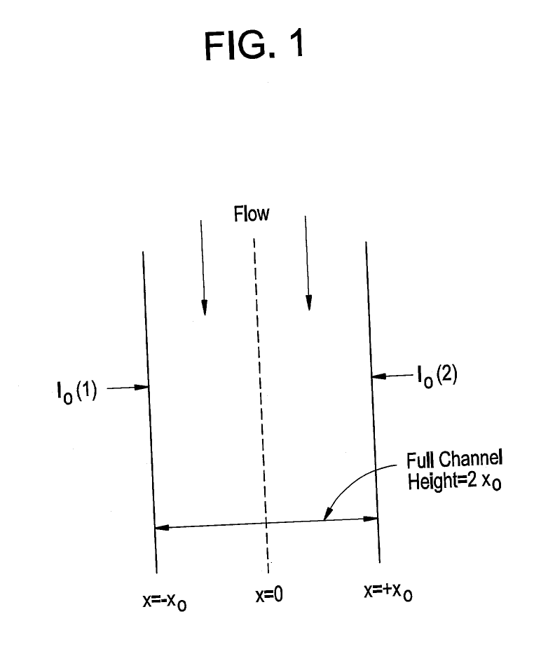

I(x)=I₀exp[−aᵤᵥ(x+x₀)]

where aᵤᵥ is Beer's law optical attenuation coefficient in a medium and x₀ is the liquid channel half-height (FIG. 1). The exposure time t(x) depends on whether the flow is laminar or fully mixed. For pressure-based laminar flow in a narrow rectangular channel (Poiseuille flow), the velocity profile is parabolic and is defined by the formula:

v(x)=v(0)[1−(x/x₀)²]

where v(0) is centerline velocity (which equals 3/2 volume-averaged velocity vₐᵥ). Similarly, t(x) is defined as follows:

t(x)=t(0)/[1−(x/x₀)²]

For flow approximating fully mixed flow, we assume each liquid element resides an equal amount of time at all channel depths and that t(x) is, therefore, a constant equal to 3/2 t(0).

From devoted UVC viral inactivation studies on small picomaviruses, we found Bᵥ (the rate constant for viral inactivation)=384 to 461 J⁻¹ cm². We determined the optical attenuation coefficient to be 73.7 cm⁻¹ for undiluted serum plasma and 23 cm⁻¹ for antihemophilic factor (AHF) concentrate. Additionally, we calculated Bₚ (the rate constant for protein modification) for Factor VIII to be 1.7 J⁻¹ cm² to 17 J⁻¹ cm².

Initial calculations were performed for laminar flow, single-sided irradiation using the following values: 2x₀=0.02 cm; I=8.3×10⁻³ W/cm²; t(0)=8 s; Fₐᵥ=0.1 J/cm²; aᵤᵥ =73.7; and Bᵥ=384 J⁻¹ cm². From these calculations, the survival fraction spatial profiles and volume-averaged values for virus and protein S_{v,p} (x) and Ŝ_{v,p}, respectively, where:

$$\hat{S} = \frac{{\int }_{- {x}_{0}}^{{x}_{0}} {S}_{v , p} ( x ) V ( x ) ⅆ x}{{\int }_{- {x}_{0}}^{{x}_{0}} V ( x ) ⅆ x}$$

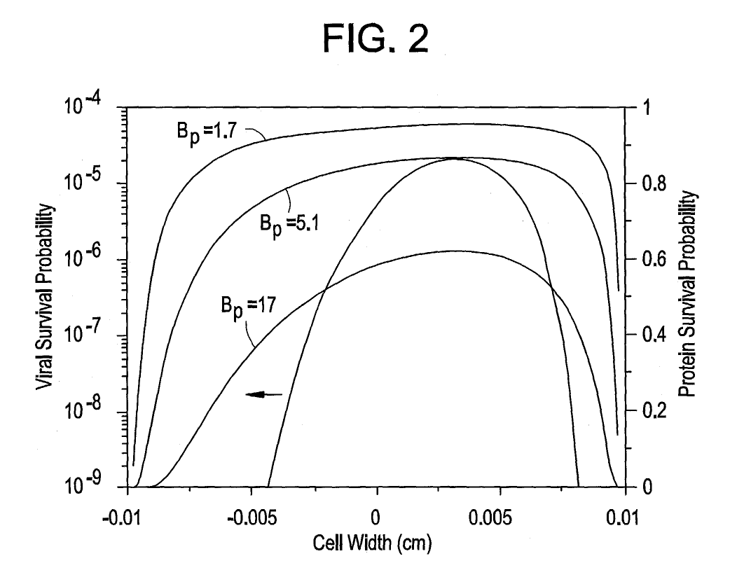

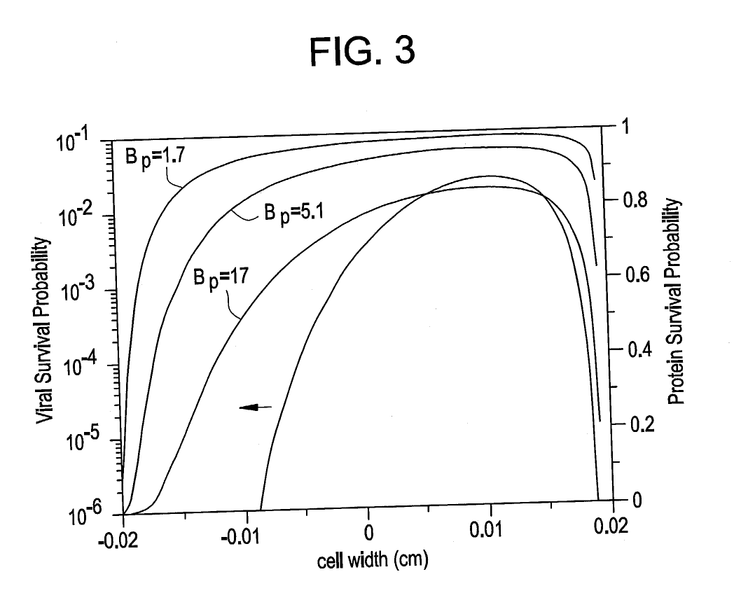

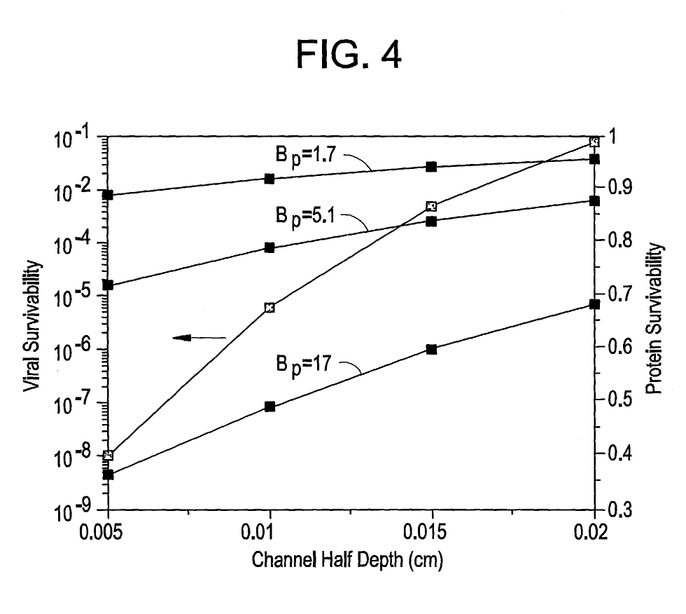

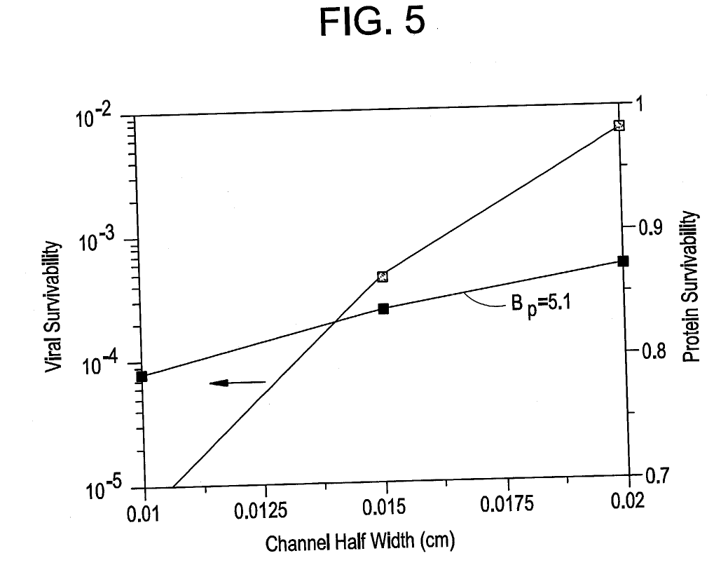

The results are shown in FIG. 2. Similar calculations were performed in which 2x₀=0.04 cm (FIG. 3), as well as 2x₀=0.03 cm and 2x₀=0.04 cm summarized in FIGS. 4 and 5.

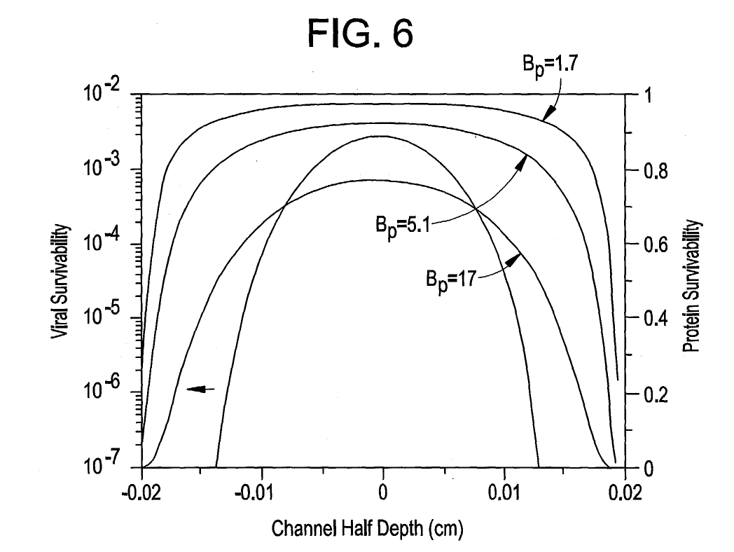

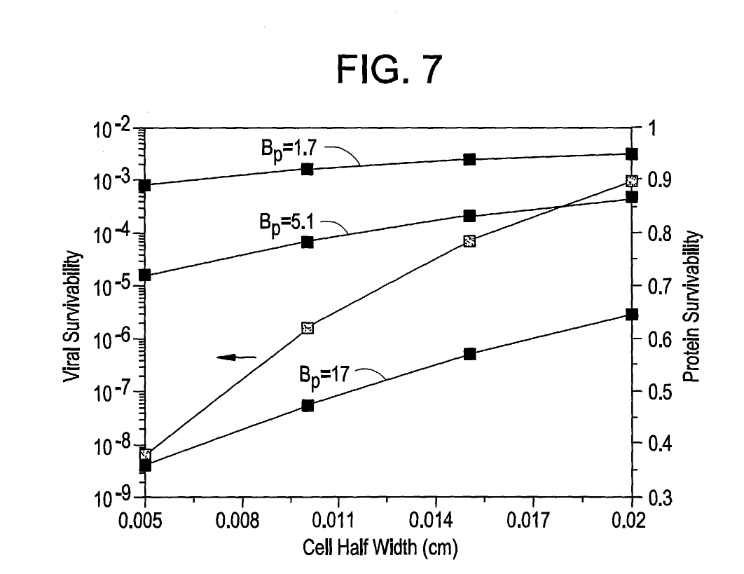

The foregoing examples utilized single-sided illumination. In another example, double-sided illumination was modeled, with each illuminator emitting half the irradiation of that in the single-sided illuminator example. Accordingly, the values were as follows: 2x₀=0.01-0.04 cm; I=2×4.15×10⁻³ W/cm²; t(0)=8 s; Fₐᵥ=0.1 J/cm²; aᵤᵥ=73.7; and Bᵥ=384 J⁻¹ cm². The results are shown in FIGS. 6-8.

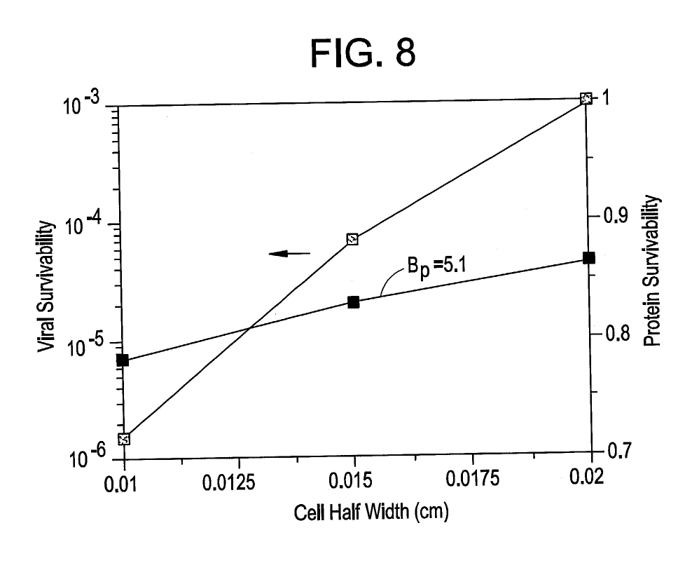

With the protein modification rate constant set at an intermediate value of 5.1 J⁻¹ cm², neither single-sided illumination nor double-sided illumination achieved the target values of >4 logs viral inactivation while maintaining >85% protein survivability (see, for example, FIGS. 5 and 8).

In the next example, viral inactivation and protein modification were calculated in a model in which approximately fully mixed flow predominated. As is described above, in such mixing, it is predicted that each liquid element resides an equal amount of time at all channel depths. This effect results in each viral particle receiving approximately the same amount irradiation. In this example, 2x₀=0.02-0.06 cm; I=8.3×10⁻³ W/cm²; t(x)=12 s; Fₐᵥ=0.1 J/cm²; aᵤᵥ=73.7; and Bᵥ=384 J⁻¹ cm². Note that the mixing allowed for a wider channel without any loss in viral inactivation. This wider channel, in turn, allows for greater throughput.

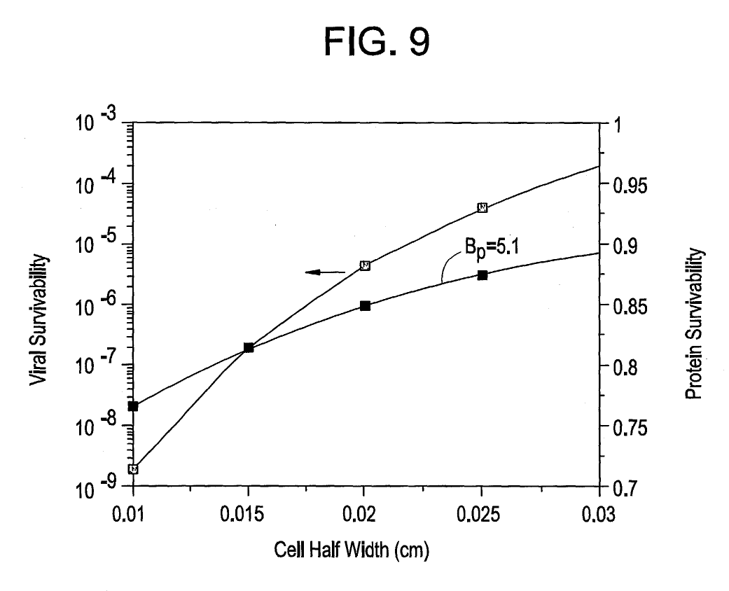

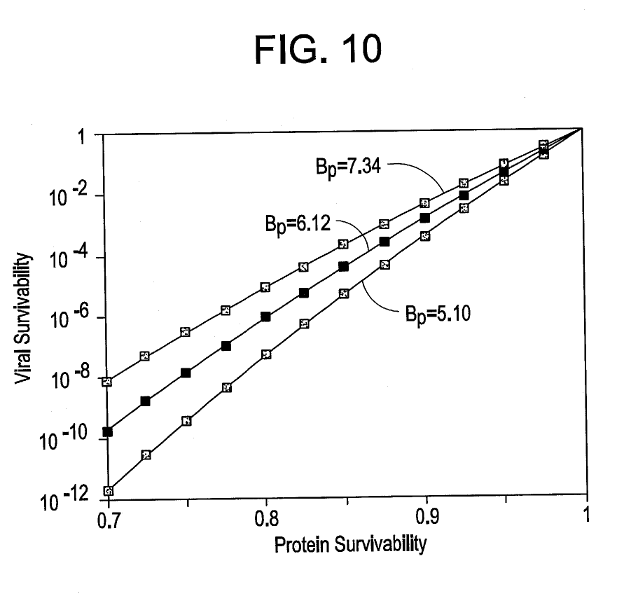

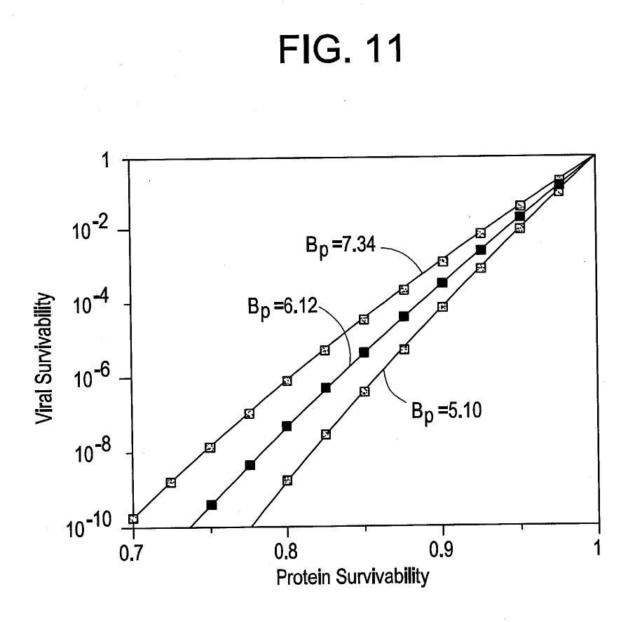

The results of the modeled viral inactivation and protein modification are depicted in FIGS. 9-11. With a Bₚ of 5.1 J⁻¹ cm², more than 85% of protein is predicted to survive while achieving a 5-log reduction in pathogen survivability (FIGS. 9 and 10).

From the foregoing data modeling, we predicted that the induction of turbulent flow prior to irradiation would allow for a high degree (>4 logs) of viral inactivation while maintaining >85% of coagulation protein activity.

Cylindrical Irradiator

We have also invented a cylindrical irradiator for the inactivation of pathogens in liquids containing biomolecules, e.g., proteins. In this embodiment, the liquid flows through a tube that is at least partially UV transparent, e.g., a quartz tube. As with flat plate irradiators, a large diameter tube allows greater throughput, but the liquid in the tube does not receive uniform irradiation. Tubes offer a simpler design than flat plates because the entry and exit ports are simply the ends of the tube, and no clamping is needed in order to assemble halves of a device. Cylindrical in-line static mixers (e.g., polyacetal mixers such as Cat. No. U-04667-12 from Cole Parmer, Vernon Hills, Ill.) may be used to mix the fluid as it flows through the tube to ensure that the liquid is more uniformaly irradiated. For these mixers, a higher flow rate results in faster mixing, as measured by completeness of mixing as a function of distance traveled down a tube (Table 1). Tubes may be employed in series or parallel to increase throughput of pathogen inactivation.

A tube may be illuminated by one lamp or surrounded by multiple lamps, e.g., two to four, to provide uniform illumination of the surface of the tube. For example, when employing one lamp, the lamp is positioned within 2-3 cm of the tube. In another example, when employing four lamps, the lamps are spaced equally around the tube and positioned within 2-7 cm of t tube. The actual position of a lamp relative to the tube and any other lamps depends, for example, on the intensity of the lamp, the number of lamps, and the desired local fluence at various positions in the tube. One skilled in the art can make this determination.

TABLE 1
Greatest distance at which at unmixed conditions can be observed
in a solution of bromophenol blue (5-mm diameter tube, mixer
has twenty-four elements per 4 ⅝ inches)

| Flow rate | Distance |
|---|---|
| (ml/mm) | (cm) |
| 2 | 8 |
| 3 | 7 |
| 4 | 6 |
| 5 | 4 |
| 6 | 3 |
| 7 | 2.5 |
| 10 | 2 |
| 20 | 1.5 |
| 40 | 1 |

Other Irradiators

One skilled in the art will recognize that other geometries, e.g., those with regular or irregular polygonal cross-sections, may be employed in the methods described herein. Any shape of irradiator is suitable as long as uniform exposure to UVC radiation can be achieved, e.g., by the inclusion of a suitable static mixer. In addition, it is possible to use different geometries, either in series or in parallel, in the same pathogen-inactivating device. As described above, the position of one or more lamps relative to each other and the liquid can be determined based on the desired local fluence and the intensity of the lamps.

Combination Treatments

The method also includes the optional step of performing solvent/detergent extraction or a chemical-mediated pathogen inactivation on the liquid. This treatment step can be performed before, during, or after irradiation. An exemplary chemical-mediated inactivation is treatment with an aziridino compound, e.g., those described in U.S. Pat. Nos. 6,093,564, 6,114,108, and 6,136,586 and U.S. Provisional Application No. ______, filed May 6, 2002, entitled “METHODS AND COMPOSITIONS FOR THE MODIFICATION OF NUCLEIC ACIDS,” each of which is hereby incorporated by reference.

A further understanding of the invention may be obtained from the following non-limiting examples.

### EXAMPLE 1

### Flat Plate Radiation Core

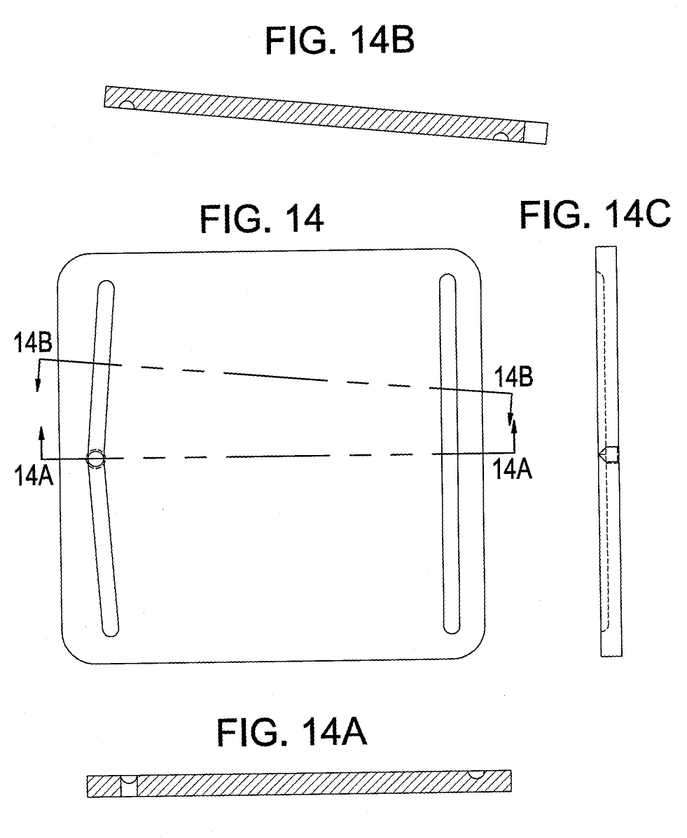

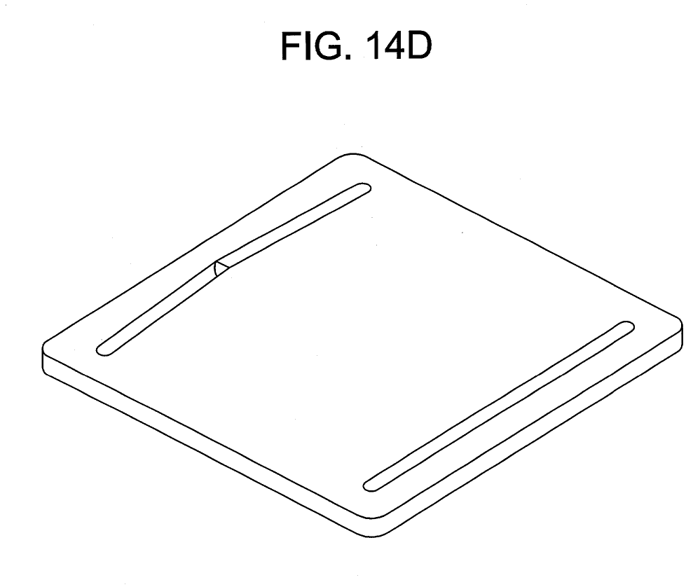

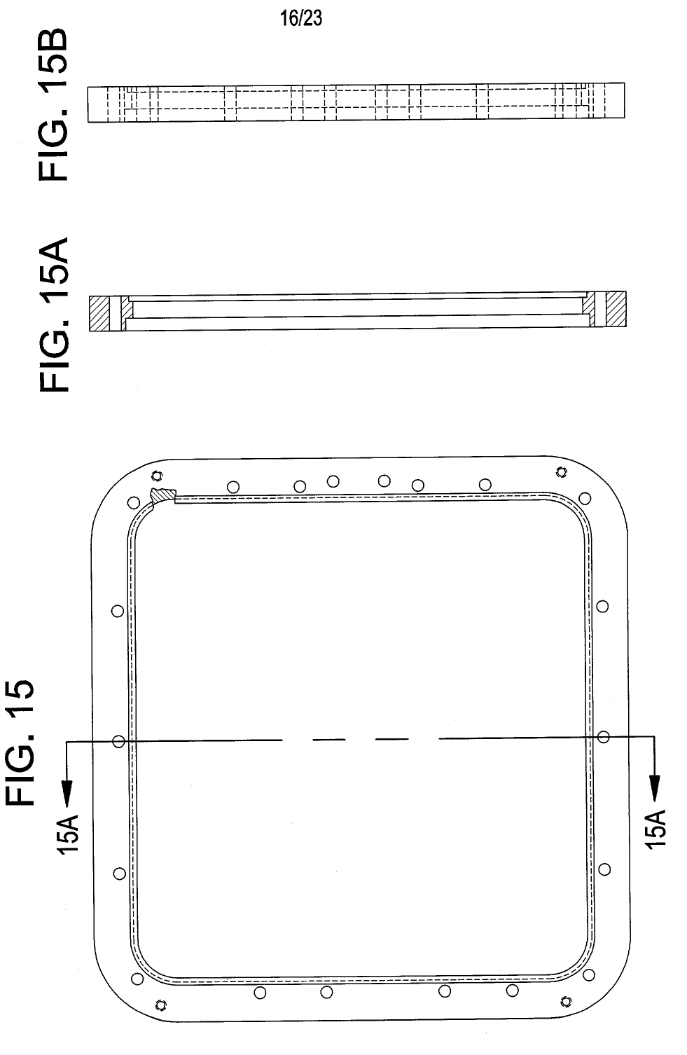

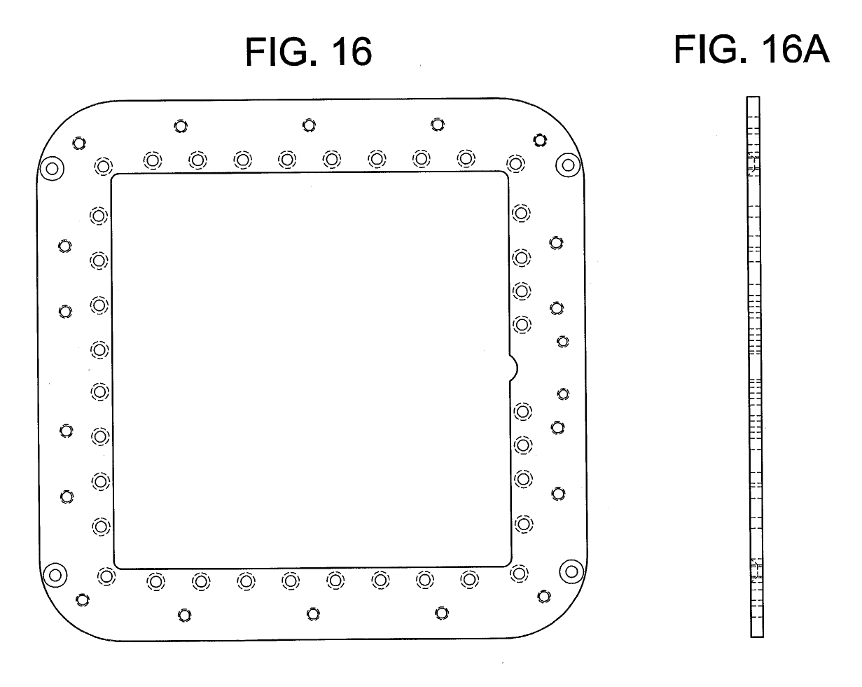

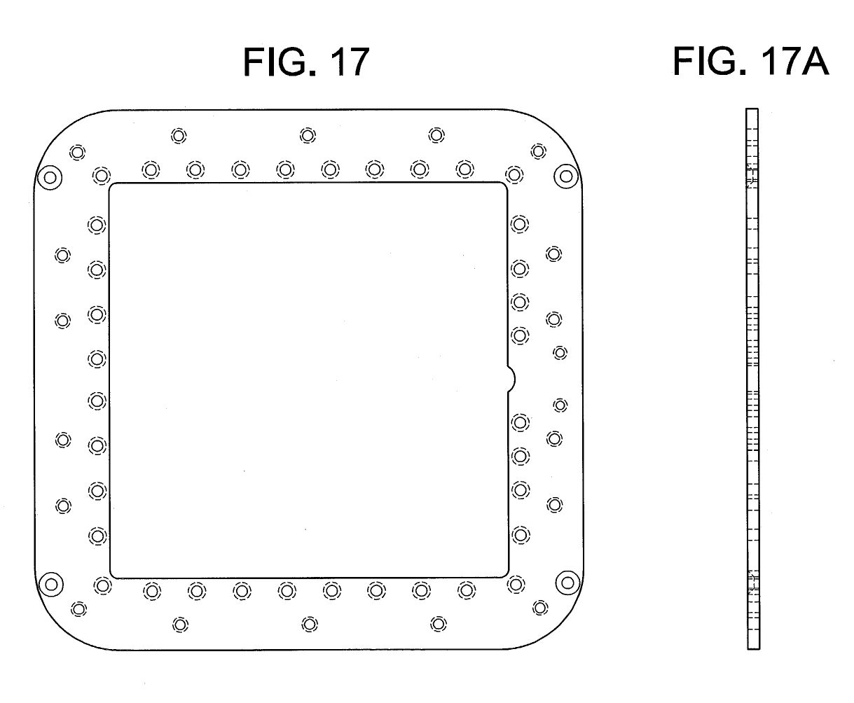

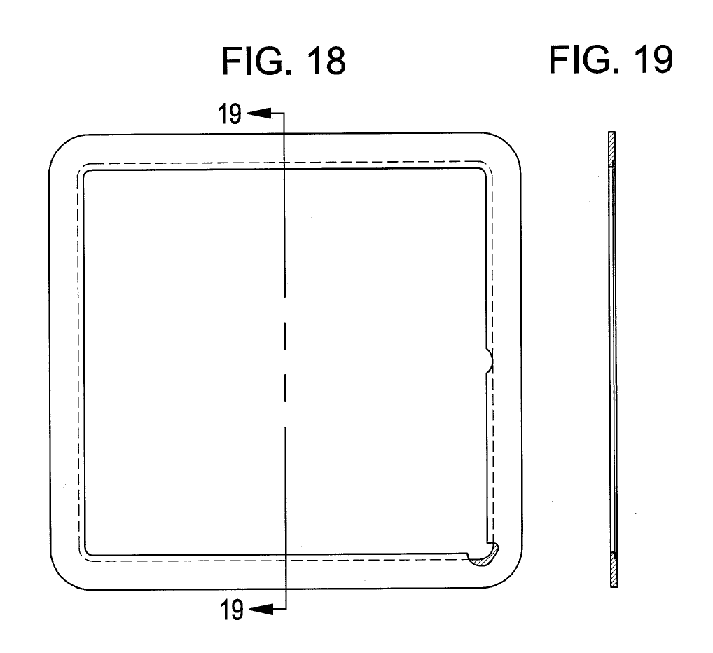

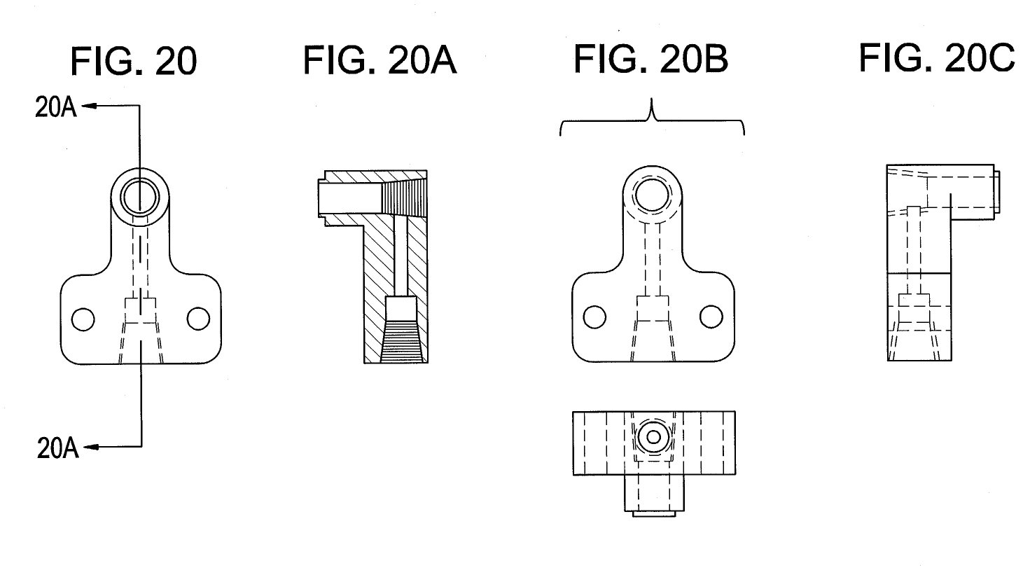

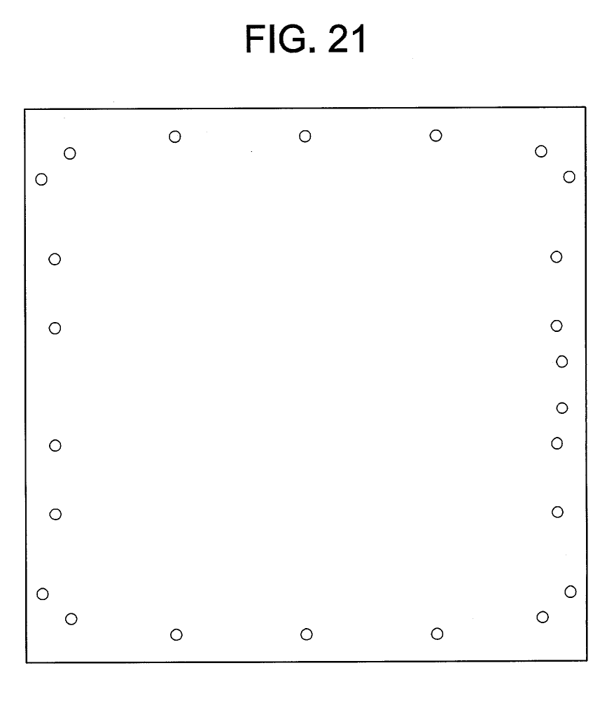

Structure. Referring to FIGS. 13-21, a radiation core is a “sandwich” assembly including two flat plates (e.g., made of quartz) clamped together with a spacer shim between. The plates were tightly clamped to create a watertight seal, and the assembly included piping ports for liquid flow in and out of the core via entry and exit connector ports.

The radiation core assembly was mounted in a dovetail slide holder inside a protective enclosure with UV lamps on both sides. Liquid was pumped into the core through the entry connector port, passed between the two quartz plates and exited via the exit connector port, which was essentially identical to the entry port. The UV lamps irradiated the liquid as it passes between the plates. Half-round cross-channels machined into the quartz plates served as liquid entry/exit manifolds. The exit cross-channel was a shallow v-shape sloped at an angle of 5° to aid air escape as liquid fills the space between the plates. The sloped channels also aided liquid drainage when the assembly was inverted.

The quartz plates were fabricated from standard UV-grade quartz, which transmitted approximately 90% of incoming radiation at 254 nm. Each plate measured 10.0″ square, and 0.5″ thick with the grooved side polished flat within 5 μm. The corners of the plates were rounded.

The quartz plates were housed inside protective retainers fabricated from nylon plastic. The opening in each retainer was slightly larger than the plate to provide clearance for differential thermal expansion or contraction. The opening of the retainer had a recess around the entire periphery for an O-ring seal, so that, when clamped, each O-ring was squeezed against the rim of the quartz plate and against the shim to form a fluid seal.

Each entry and exit connector port had two 1/8″ NPT openings for piping connections and was shaped to support an O-ring that seals against the quartz plate at the entry or exit hole machined into the plate. Each connector port was attached to a stainless steel pressure plate, with a shim between the two whose thickness was adjusted such that the O-ring was compressed slightly against the quartz plate to achieve a fluid seal.

The liquid flow channel of the radiation core was defined by a rectangular opening cut into the plastic shim between the two quartz plates. The opening can be provided with one or more screens to enhance mixing of the liquid passing through the opening. The pressure holding the two plates together was provided by spring-loaded ball plungers installed in the steel pressure plates. To avoid high contact stresses in the plates, a load spreader plate was installed between the ball plungers and the quartz plates. Further protection was provided by a thin plastic gasket inserted between the spreader plate and the quartz plates. Each of the ball plungers was adjusted to provide a spring force in the range of 4-9 lb.

Assembly. The following steps were carried out at the initial installation of the quartz plates into the core assembly, and each instance when plates were replaced.

1. If this is not the initial installation, remove the entry and exit connector port units and loosen all of the ball spring plungers, i.e., loosen the lock nuts and unscrew the plunger several turns.

2. Assemble the stainless steel pressure plates to the exposure plate retainers with the load spreader plates sandwiched between. The side of the load spreader plates that will be nearest the quartz exposure plates is provided with a thin plastic gasket or soft tape to interface against the quartz.

3. Place the pressure plate assembly with the threaded holes in a position that allows one of the quartz plates to be installed with the grooves up. The plate would be positioned with its entry/exit hole at the end where the connector port unit will be installed. This location is identifiable by the small curved cutaway on one side of the pressure plate.

4. Install the large O-ring seal around the periphery of the exposure plate. The O-ring should fit snugly into the groove between the exposure plate and the nylon retainer.

5. If not already in place, install an alignment stud into one of the threaded pressure plate clamp screw holes.

6. Install the plastic shim, taking care to align the holes in the shim with the holes in the nylon retainer and pressure plate. It is useful to drop a clamp screw into one of the holes opposite the alignment stud to maintain hole alignment during the next two steps.

7. Place the second exposure plate, grooves down, on top of the shim in alignment with the first exposure plate.

8. Install the second large O-ring around the periphery of the second exposure plate. It should fit close to the rim of the plate.

9. Remove the loose clamp screw opposite the alignment stud and carefully install the remaining pressure plate assembly using the alignment stud as a partial guide. If the second exposure plate is positioned correctly, the pressure plate assembly should slide over the exposure plate and its O-ring seal with little difficulty.

10. Install all of the pressure plate clamp screws in their holes. Do not tighten any of them until all have been fitted through the plastic shim.

11. Begin to tighten one or two clamp screws along each side of the pressure plate. By touching the gap between the nylon retainers, it is possible to sense when the gap is closing. Tighten the opposing clamp screws more or less uniformly until the gap is completely closed. During this procedure, it is useful to try moving the load spreader plates adjacent to the quartz exposure plates. The spreader plates should move easily indicating that no significant force is being applied to the exposure plates. None of the ball springs plungers should be applying forces at this point.

12. Tighten all of the pressure plate clamp screws. The nylon retainers should be firmly pressed against the shim with no gap all around.

13. Screw the top side ball plungers all the way down, one at a time, until the plunger is compressed completely, taking care not to tighten the plunger excessively. It is possible to detect when the plunger “bottoms” by a noticeable change in tightening torque. Do not torque the plunger any further. Back the plunger out about 1/4 turn, before proceeding to the next one. This leaves a small force acting on the load spreader plate.

14. Turn the core assembly over and repeat Step 13. When completed, a modest clamping force exists to press the exposure plates against both sides of the shim. Since the shim is also clamped between the nylon retainer plates it is important to keep forces more or less equal on both sides of the shim to prevent it from becoming distorted in the clamping process.

15. Tighten each of the ball plungers, one at a time, to a “bottoming” condition, then back the plunger out about 1/16 turn. Do this for both sides of the assembly. Do a final recheck on the plunger condition, then tighten the lock nuts.

16. Install the inlet and outlet connector port units with the O-ring in place. Do not tighten the attachment screws until a check is made of the amount of O-ring compression that will occur when the screws are tightened completely. The O-ring is 1/16″ thick nominally, but individual O-rings may vary in thickness. The desired amount of compression is about 0.010″. Install shims under the port unit as required to obtain the desired spacing. If necessary, a washer maybe placed behind the O-ring.

17. Internal liquid pressure may cause the exposure plates to deflect outward in operation. To reduce this deflection, crossbars may be used to exert an auxiliary clamping forced on the plates near the center. For example, two crossbars can be mounted to span across the exposure region of the plates. Each crossbar is equipped with two screws that can be extended to press against the exposure plate surface. A wooden block or other soft, stiff interface material may be used between the screws and the exposure plate to avoid scoring the plates.

### EXAMPLE 2

### Experimental Design of Flat Plate Irradiator

Before irradiation, EMCV or an equal volume of normal saline was added to the plasma solution for protein study. The mixture was pumped peristaltically through a flat plate irradiator core with internal dimensions of 51×178×0.75 mm into which was placed a static mixing device consisting of a 26 square mesh screen of stainless steel wire (Cleveland Wire Cloth Manufacturing Co., Cleveland, Ohio) having a diameter of about 0.015 inches, onto which were applied epoxy barrier strips (made from Supreme 10HTND-2; Master Bond, Inc., Hackensack, N.J.) having a width of about 0.0625 inches and spaced at intervals of about 0.15 inches. The static mixing device was oriented such that the mesh wires and the barrier strips were at a forty-five degree angle to the flow path in the apparatus in the absence of the static mixing device (FIGS. 12A and 12B). The liquid was irradiated using two-side multiple 15 watt mercury lamps emitting more than 95% energy at 254 nm. The transmission of the quartz plates was more than 90% at 254 nm. Approximately 2×7 inches of the flat plate was exposed to UVC light for about 3 to 10 seconds.

Factor V, VII, X, and XI Activity Assay

For Factor V, Factor VII, Factor X, and Factor XI determination, irradiated samples were immediately assayed or frozen at −80° C. after collection. Each sample was assayed in duplicate and the results averaged. Factor X activity and Factor XI activity were determined by one-stage activated partial thromboplastin time (APTT; Organon-Teknika Inc. Durham, N.C.) clotting assay using an automatic coagulation machine (Sysmex CA-5000; DADE International). Factor V activity and VII activity were assayed similarly, except thromboplastin with calcium replaced APTT. Factor X and XI deficient plasma was from George King Biomedical (cat. 1122-N). Factor V deficient plasma (cat. Factor V D-I) and thromboplastin with calcium (cat. 7280) were from Sigma Chemical Co.

Protein C, Protein S, Antithrombin III, Factor VIII, Factor IX and Factor XIII Assay

Protein C, Protein S, Antithrombin III, Factor VIII, Factor IX and Factor XIII was assayed for activity as measured using clotting time.

Virus Titering

EMCV and PPV titers were determined using end-point 10-fold serial dilution in 96 well microtiter plates. Virus-induced cytopathology was scored after 72 hours and viral titer were determined using standard techniques.

### EXAMPLE 3

### EMCV Inactivation of Plasma Using Flat Plate Irradiator

Using the foregoing methods, EMCV-spiked solvent/detergent (SD)-treated plasma or recovered frozen plasma was processed through the irradiator of the present invention. Flow rate was determined to be 70 ml/min, and fluence was 0.020 J/cm². An aliquot of each virus-spiked sample was collected before and after UVC irradiation. In each case, about a 4-log EMCV inactivation was achieved at a fluence of 0.020 J/cm² (Table 2).

TABLE 2

| | | | EMCV Infectivity | | |
|---|---|---|---|---|---|
| Plasma | Flow Rate | Fluence | (log) | | Virus Kill |
| type | (ml/min) | (J/cm²) | Pre-UVC | Post-UVC | (log) |
| SD-treated | 70 | 0.020 | 5.4 | 1.1 | 4.3 |
| plasma | | | | | |
| SD-treated | 70 | 0.020 | 5.1 | 1.3 | 3.8 |
| plasma | | | | | |
| Recovered | 70 | 0.020 | 6.4 | 2.6 | 3.8 |
| plasma, | | | | | |
| frozen | | | | | |

### EXAMPLE 4

### Inactivation of Porcine Parvovirus in SD-treated Plasma

Using the same conditions as described for EMCV-spiked plasma in Example 2, PPV-spiked SD-treated plasma was processed through the UVC irradiator. The amount of viral inactivation was even more than that observed with EMCV; UV irradiation inactivated PPV by an average of more than 6 logs at a fluence of about 0.017 to 0.020 J/cm² (Table 3).

TABLE 3

| Flow Rate | Fluence | PPV Infectivity (log) | | |
|---|---|---|---|---|
| (ml/min) | (J/cm²) | Pre-UVC | Post-UVC | Virus Kill (log) |
| 70 | 0.020 | 6.7 | ≦0.6 | ≧6.1 |
| 70 | 0.020 | 6.2 | 0.4 | 5.8 |
| 70 | 0.020 | 7.7 | ≦0.2 | ≧7.5 |
| 85 | 0.017 | 7.7 | ≦0.2 | ≧7.5 |

### EXAMPLE 5

### Coagulation Factor Analysis of UVC-treated SD Plasma

As described herein, an important component of pathogen inactivation in blood and blood products is the retention of the activity of coagulation factors and other proteins. Using standard assays, we measured the amount of coagulation factor recovery from SD plasma processed through the flat plate irradiator in the absence of UV irradiation (fluence=0 J/cm²). The data are shown in Table 4. In all cases, coagulation factor activity was largely unchanged.

TABLE 4

| | Coagulation factor recovery | | | | | | | |
|---|---|---|---|---|---|---|---|---|
| Flow rate | Factor V | | Factor VII | | Factor X | | Factor XI | |
| (ml/min) | Activity | Recovery | Activity | Recovery | Activity | Recovery | Activity | Recovery |
| Control | 1.16 | | 1.41 | | 1.15 | | 1.44 | |
| 50 | 1.18 | 102% | 1.46 | 104% | 1.17 | 102% | 1.41 | 98% |
| 70 | 1.20 | 103% | 1.44 | 102% | 1.15 | 100% | 1.48 | 103% |
| 85 | 1.18 | 102% | 1.43 | 101% | 1.20 | 104% | 1.46 | 101% |

We next determined the activity recovery following UVC irradiation in the flat plate irradiator with a flow rate of 70 ml/min and a fluence of 0.020 J/cm². As shown in Table 5, the percent recovery was greater than 80% for four coagulation factors.

TABLE 5

| | Coagulation recovery (for UVC irradiation alone) | | | |
|---|---|---|---|---|
| Trial | Factor V | Factor VII | Factor X | Factor XI |
| 1 | 92% | 93% | 98% | 84% |
| 2 | 91% | 81% | 82% | 84% |
| 3 | 87% | 85% | 88% | 81% |
| 4 | 95% | 94% | 98% | 82% |
| 5 | 93% | 86% | 95% | 80% |
| 6 | 92% | 93% | 92% | 85% |
| Average ± SD | 92 ± 3% | 89 ± 5% | 92 ± 6% | 83 ± 2% |

In view of the data shown in Tables 3, 4, and 5 it is readily apparent that conditions that retain at least 80% of coagulation protein activity (70 ml/min, 0.020 J/cm²) inactivate or reduce the presence of infective viruses by approximately 6-logs relative to SD plasma not UVC irradiated.

### EXAMPLE 6

### Recovery of Plasma Proteins from UVC-treated SD Plasma

In order to determine the extent that protein activity is retained, we examined the protein activity recovery for an additional seven plasma proteins following UVC treatment of SD plasma (Table 6). Flow rate was 70 ml/min, and fluence was 0.020 J/cm². The average percent recovery was greater than eighty percent for all proteins examined.

TABLE 6

| | Protein activity recovery | | | | | |
|---|---|---|---|---|---|---|
| | Trial 1 | | | Trial 2 | | |
| Plasma Protein | Pre-UVC | Post-UVC | Recovery | Pre-UVC | Post-UVC | Recovery |
| Protein C | 0.84 | 0.78 | 93% | 0.81 | 0.80 | 99% |
| Protein S | 0.74 | 0.71 | 96% | 0.63 | 0.61 | 97% |
| Alpha 2 antiplasmin | 0.16 | 0.16 | 100% | 0.19 | 0.14 | 74% |
| Antithrombin | 0.87 | 0.86 | 99% | 0.89 | 0.91 | 102% |
| Factor VIII | 0.80 | 0.69 | 86% | 0.6 | 0.53 | 88% |
| Factor IX | 0.78 | 0.63 | 81% | 0.62 | 0.57 | 92% |
| Factor XIII | 1.08 | 0.96 | 89% | 1.08 | 1.04 | 96% |

We conclude that UVC inactivation of pathogens in plasma and other blood products can be achieved with a cell height of 0.75 mm (based on the data presented herein), or even up to about 2 mm, without an unacceptable loss in protein activity, by use of a static mixing device that alters the laminar flow of the liquid through the cell to approximate that of a fully-mixed liquid.

Examples 7-12 employ a cylindrical irradiator as described herein.

### EXAMPLE 7

### Treatment of Protein Solutions

It is well known that Factor VIII, Factor IX and fibrinogen are among the most sensitive proteins to different treatment. Thus, AHF, PCC, and fibrinogen were chosen in this study to examine virucidal efficiency and its effects on protein activities. EMCV was selected as a marker virus for non-enveloped viruses because of its easy and fast detection.

Materials and methods. AHF (Mel AHF) was from Melville Biologics. Bulk fibrinogen (073196) was isolated by methods known in the art. PCC was isolated by DEAE-50 absorption in our own laboratory. UV mercury lamps were from Spectronics Corp. Uridine 5-monophosphate (UMP) was from Sigma Chemical Co. The static mixer (OD: 3/16 inches, 24 elements per 4.625 inches) was from Cole-Parmer Instrument Company.

Virus Inactivation. Before irradiation, quencher (rutin) and/or EMCV were added to the plasma proteins solutions under study. The mixture was pumped peristaltically through a quartz tube with an internal diameter of 5 mm into which was placed an opaque static mixer. The mixtures were irradiated using mercury lamps emitting more than 95% energy at 254 nm. The quartz tube was placed in the middle of several lamps.

Factor VIII and Factor IX activity. Samples for Factor VIII and Factor IX determination were frozen at −80° C. Each sample was assayed in duplicate, and the results were averaged. Factor activities were determined by one-stage activated partial thromboplastin (APTT) time clotting assay using an automatic coagulation machine (Sysmex CA-5000, Dade International). Factor deficient plasma was from George King Biomedical.

Clottable fibrinogen. Clottable fibrinogen was determined by addition of thrombin to form fibrin. Clottable proteins are the difference between total proteins and supernatant. Protein concentration was determined by standard methods.

EMCV assay. EMCV were assessed using end-point 10-fold serial dilution in a 96 well microtiter plate. Virus-induced cytopathology was scored after 72 hours of incubation at 37° C. in 5% CO₂.

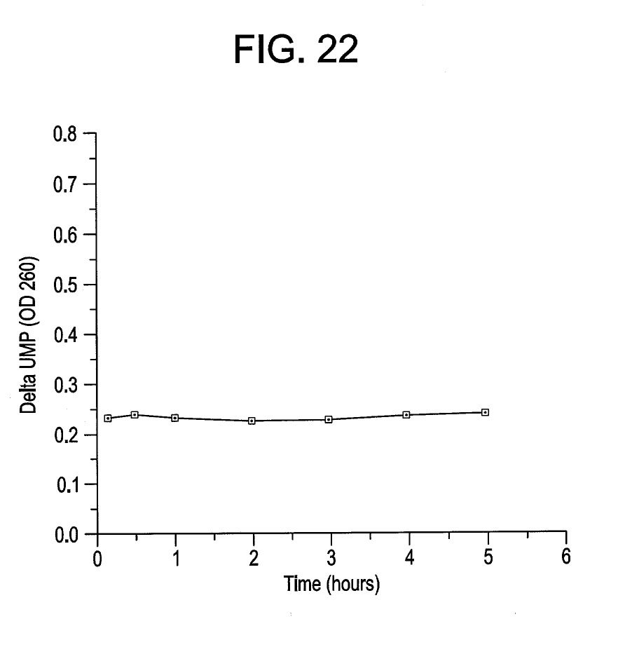

Results and discussion. UMP actinometry. UMP is broken down in proportion to UVC dosage, resulting in a decrease in absorbency at 260 nm. It was used to monitor UVC fluence. Using a single tube, the change in UMP had an inverse exponential relation to the flow rate and a direct exponential relation to the resident time. A flow rate of 15 ml/minute (resident time of 15.2 seconds) corresponded to a ΔUMP of about 0.24 (Table 7). This determination was reproducible as determined on different days and over 5-hour periods on the same day of operation. (FIG. 22). We also quantified the fluence based on measurements of irradiance made with an irradiometer placed in the middle of four lamps in a one-tube study and in three different positions in a four-tube study (Tables 7 and 8). We also investigated the change in UMP using four identical quartz tubes. The tube-to-tube determinations were also reproducible(Table 8).

TABLE 7
UMP study of static mixer system (single tube)

| Flow rate | Resident time | Fluence | ΔUMP(OD 260) | |
|---|---|---|---|---|
| (ml/min) | (second) | (J/cm²) | X ± SD (n = 3) | CV |
| 10 | 22.8 | 0.264 | 0.321 +/− 0.005 | 1.5% |
| 15 | 15.2 | 0.176 | 0.237 +/− 0.0035 | 1.48% |
| 20 | 11.4 | 0.132 | 0.185 +/− 0.0032 | 1.7% |
| 30 | 7.6 | 0.088 | 0.125 +/− 0.0025 | 2% |

Conditions: Single quartz tube, 4 lamps, 25 cm path length, intensity: 11.59 mW/cm²

TABLE 8
UMP study of static mixer system (4 tubes)

| Flow rate | Resident time | Fluence | ΔUMP(OD 260) | |
|---|---|---|---|---|
| (ml/min.) | (second) | (J/cm²) | X ± SD(n = 3) | CV |
| 60 | 15.2 | 0.139 | 0.277 +/− 0.0081 | 2.9% |
| 100 | 9.12 | 0.083 | 0.183 +/− 0.0050 | 2.7% |
| 150 | 6.1 | 0.055 | 0.124 +/− 0.0045 | 3.6% |

Conditions: 4 quartz tubes, 100-cm path length, average intensity of 3 positions: 9.13 mW/cm² (Ia = 9.6, Ib = 10.9, and Ic = 6.9)

PCC concentrate. About 85% Factor IX recovery was achieved at complete EMCV kill level (Table 9) in a four-lamp study. The Factor IX recovery was 75% with inactivation of 2.5 logs EMCV in a one-lamp study.

TABLE 9
Virucidal treatment of PCC by UVC with static mixer
system (1 tube; in the presence of 0.5 mM rutin)

| | | | Activity (u/ml) | | | |
|---|---|---|---|---|---|---|
| Flow | | Resid. | | | Factor | EMC |
| rate | Fluence | time | Pre- | Post- | IX | kill |
| (ml/min) | (J/cm²) | (second) | UVC | UVC | recovery | (log) |
| 9 | 0.243 | 25 | 9.42 | 6.13 | 65% | >5.1 |
| 12 | 0.22 | 19 | 9.42 | 7.27 | 77% | >5.1 |
| 15 | 0.176 | 15.2 | 9.42 | 8.13 | 86% | >5.1 |

Conditions: Single quartz tube, 4 lamps, 25 cm path length, intensity: 11.59 mW/cm². Total
A₂₅₄ = 16 (Aᵣᵤₜᵢₙ 10, A_{PCC} 6

AHF. About 85% Factor VIII recovery was achieved at complete EMCV kill level in a one-lamp study (Table 10). Furthermore, Factor VIII recovery was greatly enhanced by the use of four lamps. Almost 100% activity was retained at satisfactory EMCV kill levels (Table 11). Similarly to the PCC study, the Factor VIII recovery decreased with increasing fluence (Table 11).

TABLE 10
Virucidal treatment of AHF by UVC with static mixer system
(1 tube, 1 lamp in the presence of 0.5 mM rutin)

| | | | Activity (u/ml) | | | |
|---|---|---|---|---|---|---|
| Flow | | Resid. | | | Factor | EMC |
| rate | Fluence | time | Pre- | Post- | VIII | kill |
| (ml/min.) | (J/cm²) | (second) | UVC | UVC | recovery | (log) |
| 64 | 0.0258 | 3 | 29.1 | 26.2 | 90% | 1.9 |
| 32 | 0.051 | 6 | 29.1 | 21.9 | 94% | 2.9 |
| 16 | 0.103 | 12 | 29.1 | 24.2 | 86% | 4.2 |
| 8 | 0.206 | 24 | 29.1 | 27.8 | 83% | >6.2 |
| 4 | 0.412 | 48 | 29.1 | 31.3 | 86% | >6.2 |
| 2 | 0.825 | 96 | 29.1 | 19.2 | 66% | >6.2 |

Conditions: Single quartz tube, 1 lamp, 25-cm path length, intensity: 8.6 mW/cm². Total A₂₅₄ = 10 (Aᵣᵤₜᵢₙ 10, A_{AHF} <0.5)

TABLE 11
Virucidal treatment of AHF by UVC with static mixer
system (1 tube, in the presence of 0.5 mM rutin)

| | | | Activity (u/ml) | | | |
|---|---|---|---|---|---|---|
| Flow | | Resid. | | | Factor | EMC |
| rate | Fluence | time | Pre- | Post- | VIII | kill |
| (ml/min) | (J/cm²) | (second) | UVC | UVC | recovery | (log) |
| 10 | 0.264 | 22.8 | 31.1 | 21.9 | 71% | 6.5 |
| 15 | 0.176 | 15.2 | 31.1 | 24.2 | 78% | 6.5 |
| 20 | 0.132 | 11.4 | 31.1 | 27.8 | 90% | 6.5 |
| 30 | 0.088 | 7.60 | 31.1 | 31.3 | 100% | 6.5 |

Conditions: Single quartz tube, 4 lamps, 25-cm path length, intensity: 11.59 mW/cm². Total A₂₅₄ = 10 (Aᵣᵤₜᵢₙ 10, A_{AHF} <0.5)

Fibrinogen. More than 5 logs of EMCV could be killed at wide range of fluences (from 0.088 J/cm² to 0.378 J/cm²), while the biochemical properties of fibrinogen were retained, as indicated by clotting time and % clottable fibrinogen tests (Table 12). The maximum flow rate for satisfactory EMCV kill is 30 ml/min in this single tube study (Table 12).

TABLE 12
Virucidal treatment of fibrinogen by UVC with static
mixer (1 tube, in the presence of 0.5 mM rutin)

| Flow | | Resid. | Activity (u/ml) | | EMCV |
|---|---|---|---|---|---|
| rate | Fluence | Time | CT | % | kill |
| (ml/min) | (J/cm²) | (second) | (second) | Clottable | log) |
| Control | 0.000 | 00.0 | 14.6 | >97% | |
| 7 | 0.378 | 32.6 | 15 | >97% | 5.7 |
| 10 | 0.264 | 22.8 | 13.8 | >97% | 5.1 |
| 15 | 0.176 | 15.2 | 14.3 | >97% | 4.6 |
| 20 | 0.132 | 11.4 | 15 | >97% | 5.7 |
| 30 | 0.088 | 7.60 | 14.3 | >97% | 5.5 |
| 40 | 0.066 | 5.70 | 14.6 | >97% | 4.3 |
| 50 | 0.053 | 4.60 | 14.3 | >97% | 3.4 |

Conditions: Single quartz tube, 4 lamps, 25-cm path length, intensity: 11.59 mW/cm². Total A₂₅₄ = 16 (Aᵣᵤₜᵢₙ 10, A_{fibrinogen} 7) Fibrinogen: 11 mg/ml

### EXAMPLE 8

### EMCV Kill Ability vs. Transparency at 254

At the same EMCV kill level, a higher fluence was required for a solution with a lower transparency. For example, for 3 logs EMCV kill, a dose of 0.0275 J/cm² was required for a solution with A₂₅₄=20, while a dose of 0.055 J/cm² was required for a solution with A₂₅₄=40.

To examine if better virus kill and protein recovery could be achieved by improving transparency, plasma was diluted from A₂₅₄=40 to A₂₅₄=20. The results demonstrated that virus kill was increased with increasing transparency. Protein recovery, however, decreased. Thus, dilution does not appear to significantly improve protein recovery at the same virus kill level.

### EXAMPLE 9

### EMCV Inactivation of Different Proteins at Fixed Transparency

To compare EMCV inactivation ability in different protein solutions, three protein solutions, albumin, fibrinogen, and plasma, were diluted to the same absorbency, A₂₅₄=20. The results showed that, at a given fluence, EMCV in fibrinogen solutions were the most difficult to kill, while EMCV in plasma were the easiest.

### EXAMPLE 10

### Rutin Concentration

A small amount of rutin (0.1 mM) may increase protein recovery, without diminishing viral kill. Higher rutin (0.5 mM) diminishes EMCV kill by about 1 log but increases recovery of proteins by 10 to 20%, depending on the factors examined. The greatest increase was achieved with Factor VII. The least benefit was achieved with Factor V.

Among the three delicate proteins examined, Factor IX in PCC was the most sensitive to UVC. Surprisingly, fibrinogen was not sensitive to UVC treatment (Table 13).

TABLE 13
Virucidal Treatment of Fibrinogen Solution
by UVC Using In-line Static Mixer.

| UVC treatment condition: | | | Fibrinogen activity | | |
|---|---|---|---|---|---|
| Flow | | Resident | Pre-UVC | Post-UVC | EMCV |
| rate | Fluence | time | (% | (% | inactivation |
| (ml/min.) | (J/cm²) | (second) | clottable) | clottable) | (log) |
| 60 | 0.139 | 15.2 | ≧98% | ≧98% | ≧6.0 |
| 100 | 0.083 | 9.1 | ≧98% | ≧98% | ≧6.0 |
| 150 | 0.055 | 6.1 | ≧98% | ≧98% | ≧6.0 |
| 200 | 0.042 | 4.6 | ≧98% | ≧98% | ≧6.0 |

Condition: Single quartz cylinder with in-line plastic static mixer (OD: 3/16 inches, 24 elements per 4⅝ inches). Intensity: 11.59 mW/cm². 4-side illumination. A₂₅₄ = 10 (proteins plus rutin). AHF: Melville Biologies

A feature of the static mixer system was that excellent mixing was achieved by clockwise/counterclockwise and inside/outside movement of the treated solution, especially at higher flow rates. It was demonstrated that biochemical properties of PCC, AHF and fibrinogen are maintained at a satisfactory EMCV kill level. In a preferred embodiment, the cylindrical static mixer system of UVC virucidal treatment described herein is used for plasma products of protein concentrations of about 10 to 15 mg/ml or less.

### EXAMPLE 11

### Virucidal Treatment of Plasma, AHF, and PCC by Multiple Passages Through an Irradiator

Materials and methods. S/D plasma was obtained from the New York Blood Center, AHF was from Melville Biologics Inc., and PCC was prepared by DEAE A-50 absorption by standard methods. Before irradiation, quenchers (rutin, tryptophan) and EMCV were added to the protein solution under study, after which the mixture was pumped peristaltically through a quartz tube with an internal diameter of 5 mm into which was placed an opaque static mixer. The mixtures were irradiated using a mercury lamp emitting 90% of its energy at 254 nm. Total radiant energy was controlled by the flow rate. Irradiance was measured with a Spectroline DM-254 H Digital Radiometer. Coagulation factor activity was determined by one-stage activated partial thromboplastin e4 (APTT ) time clotting assay. EMCV were assessed by end-point, 10-fold serial dilutions in 96 well microtiter plates. Viral titer indicated the quantity of virus that infected 50% of tissue culture wells. After flowing through the tube (making a passage), the liquid could be collected and passed through the tube for additional passages.

Results and discussion. Complete EMCV kill was achieved after 6 passages in a single lamp study. The recovery of Factor VIII and Factor XI was, respectively, 92% and 53% (Table 14). Multiple lamps were applied in subsequent experiments (Table 15). A complete EMCV kill may be achieved after two passages (high intensity) or three passages (low intensity). The recovery of Factor VIII and Factor XI was, respectively, about 85% and 55% at complete kill level in both high intensity and low intensity studies.

TABLE 14
Virucidal treatment of plasma with UVC using a
static mixer (5 ml/min one-lamp intensity: 8.6)

| | Rutin | Tryptophan | Factor VIII | Factor VIII | Factor XI | Factor XI | EMC kill |
|---|---|---|---|---|---|---|---|
| | (mM) | (mM) | activity | recovery | activity | recovery | (log) |
| Control | | | 0.77 | | 0.97 | | |
| pre-UVC | 0.5 | 8 | 0.66 | 100 | 0.86 | 100 | 4.5 (original) |
| Passage 1 | 0.5 | 8 | 0.69 | 105 | 0.84 | 98 | 0.4 |
| 2 | 0.5 | 8 | 0.66 | 100 | 0.83 | 97 | 1.5 |
| 3 | 0.5 | 8 | 0.66 | 100 | 0.71 | 83 | 2.3 |
| 4 | 0.5 | 8 | 0.63 | 95 | 0.65 | 76 | 2.9 |
| 5 | 0.5 | 8 | 0.62 | 94 | 0.54 | 63 | 4.2 |
| 6 | 0.5 | 8 | 0.61 | 92 | 0.46 | 53 | >5.0 |

TABLE 15A
Virucidal treatment of plasma with UVC using static mixer
(4 lamp study, 0.5 mM rutin, 8 mM tryptophan)
Static mixer (6 ml/min. intensity:: 14.88 mW/cm2

| | Factor | Factor | | | |
|---|---|---|---|---|---|
| | VIII | VIII | Factor XI | Factor XI | EMC kill |
| Condition | activity | recovery | activity | recovery | (log) |
| Control | 0.84 | | 0.99 | | |
| pre-UVC | 0.72 | 100% | 0.79 | 100% | 4.6 |
| | | | | | (original) |
| Passage 1 | 0.74 | 93% | 0.61 | 77% | 2.8 |
| 2 | 0.67 | 85% | 0.45 | 57% | 5 |
| 3 | 0.61 | 82% | 0.38 | 48% | >5.1 |

TABLE 15 B
Virucidal treatment of plasma with UVC using static mixer
(4 lamp study, 0.5 mM rutin, 8 mM tryptophan)
Static mixer (6 ml/min. Intensity: 11.92 mW/cm²)

| | Factor | Factor | | | |
|---|---|---|---|---|---|
| | VIII | VIII | Factor XI | Factor XI | EMC kill |
| Condition | activity | recovery | activity | recovery | (log) |
| Control | 0.86 | 1 | | | |
| Pre-UVC | 0.81 | 100% | 0.85 | 100% | 4.3 |
| | | | | | (original) |
| Passage 1 | 0.81 | 100% | 0.74 | 87% | 1.7 |
| 2 | 0.68 | 84% | 0.56 | 66% | 3.2 |
| 3 | 0.69 | 85% | 0.46 | 54% | 4.6 |
| 4 | 0.64 | 79% | 0.35 | 41% | 4.7 |
| 5 | 0.53 | 85% | 0.28 | 33% | >4.8 |

### EXAMPLE 12

### Production Scale Irradiators

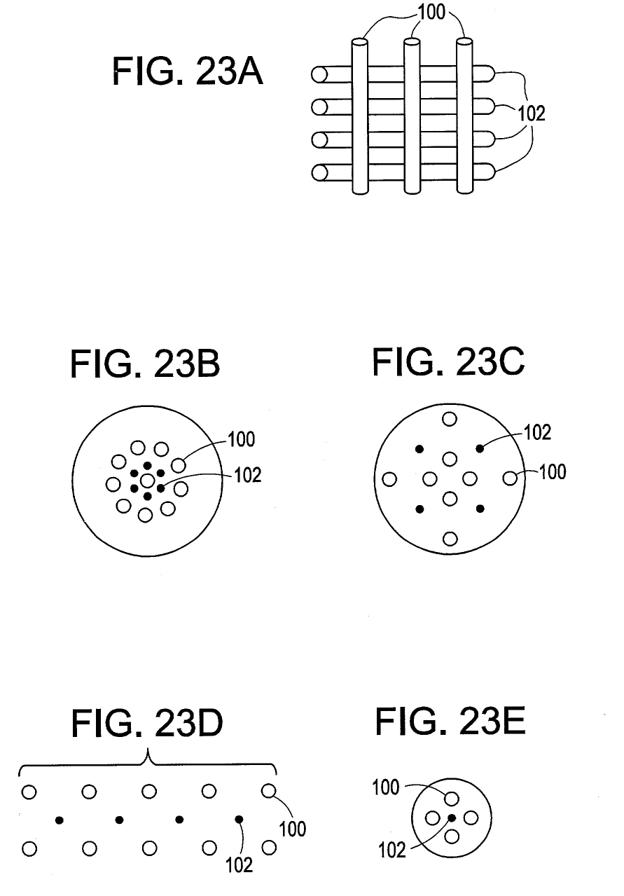

For the commercial production of pathogen-inactivated solutions, irradiators will typically process about 200 to 400 liters per hour. One method to increase throughput is to employ parallel processing of solutions. Several geometries for parallel processing of solutions by employing cylindrical irradiators are shown in FIG. 23. It is understood that variations on the number of tubes and lamps is possible and the similar designs are applicable to other irradiator geometries, e.g., flat-plate chambers. The number of chambers necessary for a given volume is easily calculated from the maximum throughput of a single chamber that achieves a desired level of pathogen inactivation. Supposing the distance between each lamp is 6 cm, 16 lamps are required for a 100-cm tube (100/6) for one-side illumination. Thirty-two lamps are required for two-side illumination. If viruses are totally killed at 200 ml/min in 100-cm tubes, the process capacity for each tube is 12 liters per hour. If 30 tubes can be illuminated by each lamp, the process capacity is 360 liters per hour for 32 lamps.

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

All publications, patents, and patent applications mentioned in this specification are herein incorporated by reference to the same extent as if each individual publication, patent, or patent application was specifically and individually to be incorporated by reference.

Other embodiments are within the claims.

What is claimed is:

1. An apparatus for treating a liquid with UV radiation to inactivate pathogens contained in the liquid, said apparatus comprising a housing comprising a liquid flow path having disposed therein a static mixing device, wherein at least a portion of said housing is radiation-transparent and adjacent a source of UV radiation that is adapted to irradiate the mixed liquid at an intensity and for a duration to inactivate pathogens therein.

2. The apparatus of claim 1, wherein said housing comprises one or more radiation-transparent plates.

3. The apparatus of claim 1, wherein said housing comprises two radiation-transparent plates, and said housing is flanked by at least two sources of UV radiation.

4. The apparatus of claim 1, wherein said housing comprises one or more radiation-transparent cylinders.

5. The apparatus of claim 1, wherein said UV radiation comprises UVC radiation.
